(19) United States
(12) Patent Application Publication
(10) Pub. No.: US 2026/0060602 A1
(43) Pub. Date: Mar. 5, 2026

(54) SYSTEM AND METHOD FOR ALLERGIC REACTION DETECTION

(71) Applicant: ANJO.AI INC, Cresskill, NJ (US)

(72) Inventors: Itamar NOCHAM, Zichron Yaakov (IL); Matan LIOR, Tzoran (IL); Tal GOLAN, Cresskill, NJ (US)

(21) Appl. No.: 19/107,254

(22) PCT Filed: Aug. 29, 2023
(86) PCT No.: PCT/IL2023/050917

§ 371 Date: Feb. 27, 2025

Related U.S. Application Data
(60) Provisional application No. 63/401,738, filed on Aug. 29, 2022.

(51) Int. Cl. A61B 5/00; A61B 5/0205; A61B 5/024; G16H 50/20

(52) U.S. Cl. CPC A61B 5/411; A61B 5/0205; A61B 5/7278; G16H 50/20; A61B 5/02405; A61B 5/02438

(57) ABSTRACT

The present disclosure provides a system and a method for predicting identifying an allergic reaction of a subject, using sensors and learning. The benefit of this system is in its ability to recognize an allergic reaction even before the visible symptoms appear. It relies on cardiovascular-related parameters, which are not difficult to generate, and can be produced by simple sensors, such as a photoplethysmography (PPG) sensor and an ECG sensor and features which are extracted from these parameters, such as heart rate variability (HRV). One of the challenges that the system of the present invention offers a solution for is handling the false positive cases, i.e., physiological reactions which may correspond with allergic reaction in terms of the measured parameters but are not in fact resulted from actual allergic reactions. This may occur, for example, when the subject is engaged in physical activity. To deal with these cases, the present invention incorporates sophisticated algorithms, which provide the ability to adjust the deviation of the parameter, and specifically HRV, from the baseline, in two time-frames scales, short-term (a time-window in the scale of minutes, e.g. 5 minutes) and long-term (a time-window that is not sensitive to, and therefore masks, momentarily or short-term peaks, such as physical exercise. The time-window is typically in the scale of about 24 hours but can be less or more than 24 hours).

### TECHNOLOGICAL FIELD

The present disclosure is in the field of wearable devices, in particular for early detection of allergic reaction.

### BACKGROUND ART

References considered to be relevant as background to the presently disclosed subject matter are listed below:
U.S. Pat. No. 9,655,532
U.S. Pat. No. 10,213,150
U.S. Pat. No. 10,799,171
Yokusoglu, M., Ozturk, S., Uzun, M., Baysan, O., Demirkol, S., Caliskaner, Z., . . . & Isik, E. (2007). Heart rate variability in patients with allergic rhinitis. Military Medicine, 172(1), 98-101.

Acknowledgement of the above references herein is not to be inferred as meaning that these are in any way relevant to the patentability of the presently disclosed subject matter.

### GENERAL DESCRIPTION

The present disclosure provides a system and a method for predicting identifying an allergic reaction of a subject, using sensors and learning. The benefit of this system is in its ability to recognize an allergic reaction even before the visible symptoms appear. It relies on cardiovascular-related parameters, which are not difficult to generate, and can be produced by simple sensors, such as a photoplethysmography (PPG) sensor and an ECG sensor and features which are extracted from these parameters, such as heart rate variability (HRV). One of the challenges that the system of the present invention offers a solution for is handling the false positive cases, i.e., physiological reactions which may correspond with allergic reaction in terms of the measured parameters but are not in fact resulted from actual allergic reactions. This may occur, for example, when the subject is engaged in physical activity. To deal with these cases, the present invention incorporates sophisticated algorithms, which provide the ability to adjust the deviation of the parameter, and specifically HRV, from the baseline, in two time-frames scales, short-term (a time-window in the scale of minutes, e.g. 5 minutes) and long-term (a time-window that is not sensitive to, and therefore masks, momentarily or short-term peaks, such as physical exercise. The time-window is typically in the scale of about 24 hours but can be less or more than 24 hours).

Therefore, an aspect of the present disclosure provides a system for identifying an allergic reaction, i.e., anaphylaxis, of a subject. The system comprising at least one sensor for sensing cardiovascular-related parameters of the subject and generate sensed data based thereon; at least one processing circuitry; one or more memories coupled to the at least one processing circuitry and storing programming instructions for execution by the at least one processing circuitry that is configured to: receiving said sensed data and calculating one or more heart rate variability (HRV) features scores, each feature score is indicative of the respective HRV feature value, namely, the value can be normalized or manipulated in any conventional method to obtain the feature score; determining whether said one or more HR V features satisfy a condition of allergic reaction; wherein if said one or more HRV features satisfy said condition, the processing circuitry is configured for generating instructions for outputting an alert. It is to be noted that the condition may be a target score of at least one HRV feature or a score of a weighted combination of two or more HRV features. Typically, in order to reduce false positive alerts, the condition is based on a score that is calculated from more than one HRV feature. The selection of the HRV features that compose the condition can be predetermined and pre-set or can be personalized and adjusted according to the subject's personal parameters, e.g. physiological parameters, medical-related parameters or other personal data (i.e. age, gender, ethnicity).

It is to be noted that any combination of the described embodiments with respect to any aspect of this present disclosure is applicable. In other words, any aspect of the present disclosure can be defined by any combination of the described embodiments.

In some embodiments of the system, the one or more HRV features scores are based on any feature that may be calculated based on R-R interval raw data that is obtained from the subject.

In some embodiments of the system, the at least one sensor comprises either a photoplethysmography (PPG) sensor, an ECG sensor, or both ECG and PPG sensors.

In some embodiments of the system, the one or more HRV features scores comprise at least one of: cardiac sympathetic index (Csi), cardiovagal index (CVi), mean R-R, or any combination thereof.

It is to be noted that any HRV feature that is described throughout the description can be used in any modification thereof, for example the Csi feature can be a modified Csi feature. Any feature can be modified and arithmetically manipulated, including, addition, subtraction, multiplication, and division with any rational number. Modifications can also include power manipulation and any polynomial function. In other words, any feature X, can also be considered as: n+X, n−X, X−n, n·X, m·Xⁿ, where n and m are any rational number (between −∞ and ∞). In some embodiments of the system, the one or more HRV features scores comprise at least one of: cardiac sympathetic index (Csi), modified Csi, standard deviation of the NN intervals (SDNN), root mean square of successive differences (RMSSD), mean heart rate, cardiovagal index (CVi), mean R-R, NN50, PNN50, NNX/PNNX (where X is less than 100 milliseconds (ms), less than 90 ms, less than 80 ms, less than 70 ms, less than 60 ms, less than 50 ms, less than 40 ms, less than 30 ms, 20 ms, or less than 10 ms), or any combination thereof.

In some embodiments of the system, the one or more HRV features scores comprise at least one of: cardiac sympathetic index (Csi), modified Csi, standard deviation of the NN intervals (SDNN), root mean square of successive differences (RMSSD), mean heart rate, cardiovagal index (CVi), mean R-R, NN50, PNN50, NN40, PNN40, NNX/PNNX (where X is 100 ms, 90 ms, 80 ms, 70 ms, 60 ms, 50 ms, 40 ms, 30 ms, 20 ms, 10 ms), the computed ratio between Csi and CVi (Ratio-SV), the multiplication of Csi and CVi (SxV), total power of density spectral, HRV variance in the Very low Frequency (VLF), HRV variance in the Low Frequency (LF), HRV variance in the High Frequency (HF), the computed ratio between LF and HF (LF-HF ratio), the normalized LF power (LFNU), or any combination thereof.

In some embodiments of the system, said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors. The at least one processing circuitry is configured to process said physiological sensed data and to affect said HRV features scores or said condition based on said physiological sensed data. Namely, the physiological sensed data is served to adjust either (1) the HRV features scores or at least some of them, (2) the condition, i.e. the threshold score(s) of a specific HRV feature or the combined weighted score of a plurality of HRV features, or (3) both the HRV features scores and the condition. For example, if high blood pressure is measured from the subject, the HRV features scores can be adjusted towards the threshold score or the condition can be adjusted to be more sensitive, namely the threshold score(s) are adjusted.

In some embodiments of the system, the processing circuitry is configured to adjust the condition based on the physiological sensed data.

In some embodiments of the system, the at least one processing circuitry is configured to identify a physiological parameter deviation indicative of a deviation or a change of at least one physiological parameter in a selected time window, wherein the selected time window is up to 10 minutes or up to 5 minutes. Upon identification of said deviation or change, the at least one processing circuitry is configured to adjust the condition of the allergy reaction to be more sensitive. In some embodiments, the physiological parameter is SPO2.

In some embodiments of the system, the condition of allergic reaction comprises an allergy reaction score calculated from said one or more HRV features scores. For example, each HRV feature may have a threshold that if the HRV feature score or value of the subject exceeds that threshold, the subject is potentially going through an allergic reaction. It is to be noted that in some embodiments, it is sufficient that only one threshold of a feature exceeds the threshold and in other embodiments there is a need of a combination of HRV features scores or values that each of them crosses its threshold, or a weighted combination of the plurality of HRV features exceeds the threshold.

In some embodiments of the system, the at least one processing circuitry is further configured for setting said condition of allergic reaction.

In some embodiments of the system, the at least one processing circuitry is configured to receive population data indicative of population condition of allergic reaction, namely HRV features scores thresholds, wherein said setting said condition of allergic reaction is performed based on said population data. In other words, the processing circuitry receives population data that indicates the allergy reaction score that the general population or population with similar characteristics as the subject have. The allergy reaction score of the population can be a starting reference point of the allergy reaction score of the subject and personalized adjustment are being made based on specific measurements that are taken from the subject.

In some embodiments of the system, the population data comprises data of population that has a selected degree of correlation with the subject. The processing circuitry may receive personal input data indicative of personal-related parameters of the subject that identify the subject and correlate the subject with a certain population to extract the relevant allergy reaction score from the population data.

In some embodiments of the system, the at least one processing circuitry is configured to generate a personalized HR V behavior data indicative of the HRV behavior profile of the subject over time by monitoring the HRV over time, wherein said setting said condition of allergic reaction is performed based on said personalized HR V behavior data and optionally also based on said population data. The personalized HRV behavior data can be segmented into anaphylaxis alert or prediction, short-term behavior, data and risk score, long-term behavior, data. The anaphylaxis alert or prediction data is defined by a time window of up to several minutes, which identifies the current HR V behavior of the subject, and the allergy reaction is typically identified in the anaphylaxis alert or prediction data, namely the anaphylaxis alert or prediction data, i.e. the short-term behavior data, is used for anaphylaxis alert/prediction. The risk score data is defined by a time window that is greater than the anaphylaxis alert or prediction, short-term behavior, data and is of between several hours and several days, which adjust the HRV baseline of the subject and therefore the condition, i.e. the allergy reaction score of the subject, if a deviation of the typical HRV baseline of the subject is identified. For example, when the subject is ill or in stress, the HRV baseline can be different than the normal HRV of the subject, indicating that he/she may be prone to an allergic reaction more than usual. Therefore, the allergy reaction score may be adjusted based on the risk score data. It is to be noted that additional physiological parameters may contribute the risk score, long-term behavior data, such as body temperature, glucose levels, blood saturation, histamine levels, etc.

It is to be noted that the condition of allergic reaction may be determined by either a preset condition that is retrieved from statistical data of the population or can be personalized through time based on (i) measurements of HRV features of the subject, (ii) input of a user regarding to alerts of allergic reaction that are identified by the at least one processing circuitry and being output to the subject or a user of the system, or (iii) both measurements of HRV features and input of a user. The user may be the subject or any related person to the subject that has the access to the system that being used by the subject.

In some embodiments of the system, said setting comprises applying varying weight factors on the population data and the personalized HRV behavior data. Namely, through time, when more personalized data is collected and available, its weight in the setting of the condition of allergic reaction increases and the population data weight decreases. In some embodiments, the weight factor of the population data may be between 1-0 on the scale of 1-0, namely it may be that the population data determines solely the condition or that the personalized HRV data determines solely the condition or any weighted combination thereof.

In some embodiments of the system, the at least one processing circuitry is configured to define HR V baseline based on said personalized HRV behavior data. HRV baseline is a typical HRV range of the subject, wherein the processing circuitry is configured to identify in said personalized HRV behavior data a long-term deviation of HRV baseline, wherein upon identification of said long term deviation of HRV baseline, the processing circuitry is further configured to perform at least one of: (i) generating instructions for outputting a high risk alert for an allergic reaction, (ii) setting said condition of allergic reaction to be more sensitive for alerts for a selected period of time or until no long term deviation is identified, namely, adjusting the score of the HRV features that triggers an alert (namely making the condition of triggering an alert to be more sensitive), or (iii) a combination of (i) and (ii). Long-term deviation is defined by a time-window frame that is greater than the short-term algorithm, for example of at least 5 hours, 10 hours, 24 hours, 48 hours or at least 72 hours, and should be understood as a deviation that is not affected by potential short-term activities of the subject, such as physical activity. The short-term deviation is defined by a time-window frame of about several minutes, namely up to 2 minutes, 3 minutes, 4 minutes, 5 minutes, 6 minutes, 7 minutes, 8 minutes, 9 minutes, 10 minutes, 15 minutes, 20 minutes or up to 30 minutes.

In some embodiments the system comprising an accelerometer for generating acceleration data indicative of the movement of the subject, wherein the personalized HRV behavior data is generated also based on the acceleration data. Namely, the personalized HR V behavior data may include the acceleration data that may indicate time windows in which the subject is doing any physical activity. The processing circuitry is configured to use the acceleration data to adjust the condition, i.e. the allergy reaction score that is constituted by one or more HRV features scores, in the time windows that are tagged as correlated with physical activity to be less sensitive. For example, the acceleration data may indicate if the subject is in movement that can affect his/her HRV so as to avoid false positive alerts of allergic reactions.

In some embodiments of the system, the personalized HRV behavior data comprises historical sensed data of the subject. The historical sensed data can be data collected by the system or imported from historical records of the subject.

In some embodiments of the system, the condition of allergic reaction comprises a score threshold of at least Csi feature, or the modified Csi feature.

In some embodiments of the system, the condition of allergic reaction comprises a score threshold of at least one of: mean R-R feature and CVi feature or a combination thereof.

In some embodiments of the system, the condition of allergic reaction comprises a score threshold of weighted calculations of two or more of: Csi feature or modified Csi feature, mean R-R feature and CVi feature.

In some embodiments the system comprising a limb accelerometer, which can be the same accelerometer that measures the general movement of the subject, for generating limb acceleration data indicative of the movement of a limb of the subject, wherein the processing circuitry is configured to identify a signature pattern in the limb acceleration data indicative of a certain action or activity, and upon identifying the signature pattern, generating instructions to increase sampling rate of the at least one sensor for a selected time period, to ensure that sufficient sensed data is received while the subject is performing said action or activity and may be in a higher risk for an allergic reaction. For example, the signature pattern can be indicative of an active motion of the subject, such as eating, or passive motion of the subject, such as falling or sleeping. These certain actions or activities are identified as ones that require to increase to sampling rate of the sensors as the risk for allergy reaction increases if they are identified. The increase in the sampling rate may also be accompanied by a short-term adjustment of the allergy reaction score.

In some embodiments of the system, the signature pattern is indicative of eating. Since eating periods are the most dangerous times, when the system identifies that the subject is eating, the sampling rate from the relevant sensors increases.

In some embodiments of the system, said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors. The processing circuitry is configured to analyze said physiological sensed data to identify an abnormality or a deviation of one or more physiological parameters that are comprised in the physiological sensed data. Upon identification of abnormality or a deviation of one or more physiological parameters, the processing circuitry is configured to generate instructions to increase sampling rate of the at least one sensor for a selected time period.

Another aspect of the present disclosure provides a method for identifying an allergic reaction, i.e., anaphylaxis, of a subject, comprising: receiving sensed data, wherein said sensed data is generated from at least one sensor for sensing cardiovascular-related parameters of the subject; calculating one or more heart rate variability (HRV) features scores, each feature score is indicative of the respective HRV feature value, namely, the value can be normalized or manipulated in any conventional method to obtain the feature score; and determining whether said one or more HRV features satisfy a condition of allergic reaction; wherein if said one or more HRV features satisfy said condition, the processing circuitry is configured for generating instructions for outputting an alert.

In some embodiments of the method, the at least one sensor comprises photoplethysmography (PPG) sensor, electrocardiogram (ECG) sensor or both.

In some embodiments of the method, the at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors; wherein the method further comprises processing said physiological sensed data and adjusting said HRV features scores based on said physiological sensed data.

In some embodiments, the method further comprises identifying a physiological parameter deviation indicative of a deviation or a change of at least one physiological parameter in a selected time window, wherein the selected time window is up to 10 minutes or up to 5 minutes. Upon identification of said deviation or change, the method further comprises adjusting the condition of the allergy reaction to be more sensitive. In some embodiments, the physiological parameter is SPO2. In some embodiments of the method, the one or more heart rate variability (HRV) features scores comprise at least one of: cardiac sympathetic index (Csi), modified Csi, standard deviation of the NN intervals (SDNN), root mean square of successive differences (RMSSD), mean heart rate, cardiovagal index (CVi), mean R-R, NN50, PNN50, NNX/PNNX (where X is 100 ms, 90 ms, 80 ms, 70 ms, 60 ms, 50 ms, 40 ms, 30 ms, 20 ms, 10 ms), or any combination thereof.

In some embodiments of the method, the one or more HRV features scores are based on any feature that may be calculated based on R-R interval raw data that is obtained from the subject.

In some embodiments of the method, the one or more HRV features scores comprise at least one of: cardiac sympathetic index (Csi), modified Csi, standard deviation of the NN intervals (SDNN), root mean square of successive differences (RMSSD), mean heart rate, cardiovagal index (CVi), mean R-R, NN50, PNN50, NN40, PNN40, NNX/PNNX (where X is less than 100 ms, less than 90 ms, less than 80 ms, less than 70 ms, less than 60 ms, less than 50 ms, less than 40 ms, less than 30 ms, less than 20 ms, less than 10 ms), the computed ratio between Csi and CVi (Ratio-SV), the multiplication of Csi and CVi (SxV), total power of density spectral, HRV variance in the Very low Frequency (VLF), HRV variance in the Low Frequency (LF), HRV variance in the High Frequency (HF), the computed ratio between LF and HF (LF-HF ratio), the normalized LF power (LFNU), or any combination thereof.

In some embodiments of the method, the condition of allergic reaction comprises an allergy reaction score. For example, each HRV feature may have a threshold that if the HRV feature score or value of the subject exceeds that threshold, the subject is potentially going through an allergic reaction. It is to be noted that in some embodiments, it is sufficient that only one threshold of a feature is exceeded and in other embodiments there is a need of a combination of HRV features scores or values that each of them crosses its threshold.

In some embodiments, the method comprises setting the condition of allergic reaction.

In some embodiments, the method comprises receiving population data indicative of population condition of allergic reaction, namely HRV features scores thresholds, wherein said setting is performed based on said population data.

In some embodiments of the method, the population data comprises data of population that has a selected degree of correlation with the subject. The method may further comprise receiving personal input data indicative of personal-related parameters of the subject that identify the subject and correlate the subject with a certain population.

In some embodiments, the method comprises generating a personalized HRV behavior data indicative of the HRV behavior profile of the subject over time by monitoring the HRV over time, wherein said setting is performed based on said personalized HRV behavior data and said population data.

In some embodiments of the method, said setting comprises applying varying weight factors on the population data and the personalized HRV behavior data. Namely, through time, when more personalized data is collected and available, its weight in the setting of the condition of allergic reaction increases and the population data weight decreases. In some embodiments, the weight factor of the population data may be between 1-0 in the scale of 1-0.

In some embodiments, the method comprises defining HRV baseline based on said personalized HRV behavior data. HRV baseline is a typical HRV range of the subject. The personalized HRV behavior data can be segmented into anaphylaxis alert or prediction, short-term behavior, data and risk score, long-term behavior, data. The anaphylaxis alert or prediction data is defined by a time window of up to several minutes, which identifies the current HRV behavior of the subject, and the allergy reaction is typically identified in the anaphylaxis alert or prediction data, namely the anaphylaxis alert or prediction data, i.e. the short-term behavior data, is used for anaphylaxis alert/prediction. The risk score data is defined by a time window that is greater than the anaphylaxis alert or prediction, short-term behavior, data and is of between several hours and several days, which adjust the HRV baseline of the subject and therefore the condition, i.e. the allergy reaction score of the subject, if a deviation of the typical HRV baseline of the subject is identified. It is to be noted that additional physiological parameters may contribute the risk score, long-term behavior data, such as body temperature, glucose levels, blood saturation, histamine levels, etc.

The method further comprises identifying in said personalized HRV behavior data a long term deviation from the HRV baseline, wherein upon identification of said long term deviation of HRV baseline, the method further comprises performing at least one of: (i) generating instructions for outputting a high risk alert for an allergic reaction, (ii) setting said condition of allergic reaction to be more sensitive for alerts for a selected period of time or until no long term deviation is identified, namely, adjusting (e.g. decreasing) the score of the HRV features that triggers an alert, or (iii) a combination of (i) and (ii). Long term deviation should be understood as a deviation that is identified in a significant period of time in the risk score data. For example, the significant period of time can be defined as a time period that is greater than a selected threshold with respect to the time in which the deviation is not observed in the selected time window.

In some embodiments of the method, the personalized HRV behavior data comprises historical sensed data of the subject.

In some embodiments of the method, the condition of allergic reaction comprises a score threshold of at least modified Csi feature.

In some embodiments of the method, the condition of allergic reaction comprises a score threshold of at least one of: mean R-R feature and CVi feature or a combination thereof.

In some embodiments of the method, the condition of allergic reaction comprises a score threshold of weighted calculations of two or more of: modified Csi feature, mean R-R feature and CVi feature.

In some embodiments, the method further comprises receiving or generating limb acceleration data indicative of the movement of a limb of the subject. The method further comprises identifying a signature pattern in the limb acceleration data indicative of a certain action or activity, and upon identifying the signature pattern, generating instructions to increase sampling rate of the at least one sensor for a selected time period, to ensure that sufficient sensed data is received while the subject is performing said action or activity and may be in a higher risk for an allergic reaction. The increase in the sampling rate may also be accompanied by adjusting the allergy reaction for a short-term.

In some embodiments of the method, the signature pattern is indicative of eating. Since eating periods are the most dangerous times, when eating activity of the subject is identified, the sampling rate from the relevant sensors increases.

In some embodiments of the method, said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors. The method further comprises analyzing said physiological sensed data to identify an abnormality or a deviation of one or more physiological parameters that are comprised in the physiological sensed data. Upon identification of abnormality or a deviation of one or more physiological parameters, the method further comprises increasing the sampling rate of the at least one sensor for a selected time period.

### EMBODIMENTS

The following are optional embodiments and combinations thereof in accordance with aspects of the present disclosure:
1. A system for identifying an onset of a chronic condition, typically a condition that involves a rapid onset of physiological reaction, of a subject, comprising:
   at least one sensor for sensing cardiovascular-related parameters of the subject and generate sensed data based thereon;
   at least one processing circuitry; one or more memories coupled to the at least one processing circuitry and storing programming instructions for execution by the at least one processing circuitry that is configured to:
   receiving said sensed data and calculating one or more heart rate variability (HRV) features scores, each feature score is indicative of the respective HRV feature value;
   determining whether said one or more HR V features satisfy a condition of an onset of a chronic physiological reaction;
   wherein if said one or more HRV features satisfy said condition, the processing circuitry is configured for generating instructions for outputting an alert.
2. The system of embodiment 1, wherein said at least one sensor comprises photoplethysmography (PPG) sensor, electrocardiogram (ECG) sensor or both.
3. The system of embodiment 1 or 2, wherein said one or more heart rate variability (HRV) features scores comprise at least one of: cardiac sympathetic index (Csi), modified Csi, standard deviation of the NN intervals (SDNN), root mean square of successive differences (RMSSD), mean heart rate, cardiovagal index (CVi), mean R-R, NN50, PNN50, or any combination thereof.
4. The system of any one of embodiments 1-3, wherein said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors;
   wherein said at least one processing circuitry is configured to process said physiological sensed data and to affect said HRV features scores based on said physiological sensed data.
5. The system of any one of embodiments 1-4, wherein said condition of an onset of chronic reaction comprises a chronic reaction score.
6. The system of any one of embodiments 1-5, wherein said at least one processing circuitry is further configured for setting said condition of an onset of chronic reaction.
7. The system of embodiment 6, wherein said at least one processing circuitry is configured to receive population data indicative of population condition of said chronic reaction, wherein said setting is performed based on said population data.
8. The system of embodiment 6, wherein the population data comprises data of population that has a selected degree of correlation with the subject.
9. The system of embodiment 7 or 8, wherein said processing circuitry is configured to generate a personalized HRV behavior data indicative of the HRV behavior profile of the subject over time, wherein said setting is performed based on said personalized HRV behavior data and said population data.
10. The system of embodiment 9, wherein said setting comprises applying varying weight factors on the population data and the personalized HRV behavior data.
11. The system of embodiment 9 or 10, wherein the processing circuitry is configured to define HRV baseline based on said personalized HRV behavior data, wherein the processing circuitry is configured to identify in said personalized HRV behavior data a long term deviation of HRV baseline, wherein upon identification of said long term deviation of HRV baseline, the processing circuitry is further configured to perform at least one of: (i) generating instructions for outputting a high risk alert for an onset of chronic reaction, (ii) setting said condition of an onset of chronic reaction to be more sensitive for alerts for a selected period of time or until no long term deviation is identified, or (iii) a combination of (i) and (ii).
12. The system of any one of embodiments 9-11, comprising an accelerometer for generating acceleration data indicative of the movement of the subject, wherein the personalized HR V behavior data is generated also based on the acceleration data.
13. The system of any one of embodiments 9-12, wherein said personalized HRV behavior data comprises historical sensed data of the subject.
14. The system of any one of embodiments 1-13, wherein said condition of an onset of chronic reaction comprises a score threshold of at least modified Csi feature.
15. The system of any one of embodiments 1-14, wherein said condition of an onset of chronic reaction comprises a score threshold of at least one of: mean R-R feature and CVi feature or a combination thereof.
16. The system of any one of embodiments 1-15, wherein said condition of an onset of chronic reaction comprises a score threshold of weighted calculations of two or more of: modified Csi feature, mean R-R feature and CVi feature.
17. The system of any one of embodiments 1-16, comprising a limb accelerometer for generating limb acceleration data indicative of the movement of a limb of the subject, wherein the processing circuitry is configured to identify signature pattern in the limb acceleration data indicative of a predefined motion or action of the subject, and upon identifying the signature pattern, generating instructions to increase sampling rate of the at least one sensor for a selected time period.
18. The system of embodiment 17, wherein said signature pattern is indicative of eating activity of the subject.
19. The system of any one of embodiments 1-18, wherein said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors;
   wherein the processing circuitry is configured to analyze said physiological sensed data to identify an abnormality or a deviation of one or more physiological parameters that are comprised in the physiological sensed data;
   wherein upon identification of abnormality or a deviation of said one or more physiological parameters, the processing circuitry is configured to generate instructions to increase sampling rate of the at least one sensor for a selected time period.
20. The system of any one of embodiments 1-19, wherein the chronic condition is a nervous system-related condition.
21. The system of any one of embodiments 1-20, wherein the chronic condition is asthma or epilepsy.
22. A method for identifying an onset of a chronic condition, typically a condition that involves a rapid onset of physiological reaction, of a subject, comprising:
   receiving sensed data, wherein said sensed data is generated from at least one sensor for sensing cardiovascular-related parameters of the subject;
   calculating one or more heart rate variability (HRV) features scores, based on said sensed data, each feature score is indicative of the respective HRV feature value; and
   determining whether said one or more HRV features satisfy a condition of an onset of chronic reaction;
   wherein if said one or more HRV features satisfy said condition, the processing circuitry is configured for generating instructions for outputting an alert.
23. The method of embodiment 22, wherein said at least one sensor comprises photoplethysmography (PPG) sensor, electrocardiogram (ECG) sensor or both.
24. The method of embodiments 22 or 23, wherein said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors;
   wherein the method further comprises processing said physiological sensed data and affecting said HRV features scores based on said physiological sensed data.
25. The method of any one of embodiments 22-24, wherein said one or more heart rate variability (HRV) features scores comprise at least one of: cardiac sympathetic index (Csi), modified Csi, standard deviation of the NN intervals (SDNN), root mean square of successive differences (RMSSD), mean heart rate, cardiovagal index (CVi), mean R-R, NN50, PNN50, or any combination thereof.
26. The method of any one of embodiments 22-25, wherein said condition of an onset of chronic reaction comprises a chronic reaction score.
27. The method of any one of embodiments 22-26, further comprising setting said condition of an onset of chronic reaction.
28. The method of embodiment 27, further comprising receiving population data indicative of population condition of said chronic reaction, wherein said setting is performed based on said population data.
29. The method of embodiment 28, wherein the population data comprises data of population that has a selected degree of correlation with the subject.
30. The method of embodiment 28 or 29, further comprising generating a personalized HRV behavior data indicative of the HRV behavior profile of the subject over time, wherein said setting is performed based on said personalized HRV behavior data and said population data.
31. The method of embodiment 30, wherein said setting comprises applying varying weight factors on the population data and the personalized HRV behavior data.
32. The method of embodiment 30 or 31, further comprising defining HRV baseline based on said personalized HRV behavior data, wherein the method further comprises identifying in said personalized HRV behavior data a long term deviation of HRV baseline, wherein upon identification of said long term deviation of HRV baseline, the method further comprises performing at least one of: (i) generating instructions for outputting a high risk alert for an onset of chronic reaction, (ii) setting said condition of an onset of chronic reaction to be more sensitive for alerts for a selected period of time or until no long term deviation is identified, or (iii) a combination of (i) and (ii).
33. The method of any one of embodiments 30-32, wherein said personalized HRV behavior data comprises historical sensed data of the subject.
34. The method of any one of embodiments 22-33, wherein said condition of an onset of chronic reaction comprises a score threshold of at least modified Csi feature.
35. The method of any one of embodiments 22-34, wherein said condition of an onset of chronic reaction comprises a score threshold of at least one of: mean R-R feature and CVi feature or a combination thereof.
36. The method of any one of embodiments 22-35, wherein said condition of an onset of chronic reaction comprises a score threshold of weighted calculations of two or more of: modified Csi feature, mean R-R feature and CVi feature.
37. The method of any one of embodiments 22-36, comprising receiving or generating limb acceleration data indicative of the movement of a limb of the subject;
   wherein the method further comprises identifying a signature pattern in the limb acceleration data indicative of a certain action or activity, and upon identifying the signature pattern, generating instructions to increase sampling rate of the at least one sensor for a selected time period.
38. The method of embodiment 37, wherein the signature pattern is indicative of eating activity.
39. The method of any one of embodiments 22-38, wherein said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors;
   wherein the method further comprises analyzing said physiological sensed data to identify an abnormality or a deviation of one or more physiological parameters that are comprised in the physiological sensed data;
   wherein upon identification of abnormality or a deviation of one or more physiological parameters, the method further comprises increasing the sampling rate of the at least one sensor for a selected time period.
40. The method of any one of embodiments 22-39, wherein the chronic condition is a nervous system-related condition.
41. The method of any one of embodiments 22-40, wherein the chronic condition is asthma or epilepsy.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a block diagram exemplifying a non-limiting embodiment of the system according to an aspect of the present disclosure.

FIG. 2 is a flow diagram exemplifying a non-limiting embodiment of the method according to an aspect of the present disclosure.

FIG. 3 is a table describing some of the relevant HR V-related features.

FIG. 4 is a schematic illustration of a block diagram exemplifying the blending model according to the present disclosure.

FIG. 5 is a schematic illustration exemplifying the distribution of the components of the system in a non-limiting embodiment.

FIG. 6 is a schematic illustration exemplifying the long-term algorithm according to the present disclosure.

FIG. 7 is a schematic illustration exemplifying the setting of the allergic reaction score based on all input and collected data and population data.

### DETAILED DESCRIPTION

The following figures are provided to exemplify embodiments and realization of the invention of the present disclosure.

The term “about” should be interpreted as a deviation of ±20% of the nominal value. For example, if the value is about 10, thus it should be understood to be in the range of 8-12.

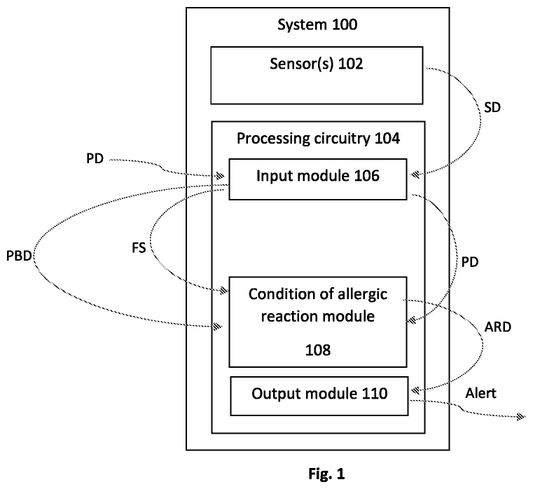

FIG. 1 is a block diagram of exemplary embodiments of the system of the present disclosure. FIG. 1 exemplifies a system 100 for identifying an allergic reaction, i.e., anaphylaxis, of a subject. The system 100 comprises at least one sensor 102, which may comprise a photoplethysmography (PPG) sensor, an ECG sensor or both PPG and ECG sensors, for sensing cardiovascular-related parameters of the subject, and generate sensed data SD based thereon. The at least one sensor 102 may also comprise an accelerometer for generating acceleration data, indicative of the movement of the subject, that is comprised in the sensed data SD. The accelerometer is configured to sense acceleration of the subject and also acceleration of the hand or wrist of the subject on which it is worn. Other types of sensors which may be included in the system 100 are: electrocardiogram (ECG) sensor, temperature sensor for measuring skin temperature of the subject, accelerometer, blood pressure sensor, blood histamine level sensor, rush detector and sweat detector.

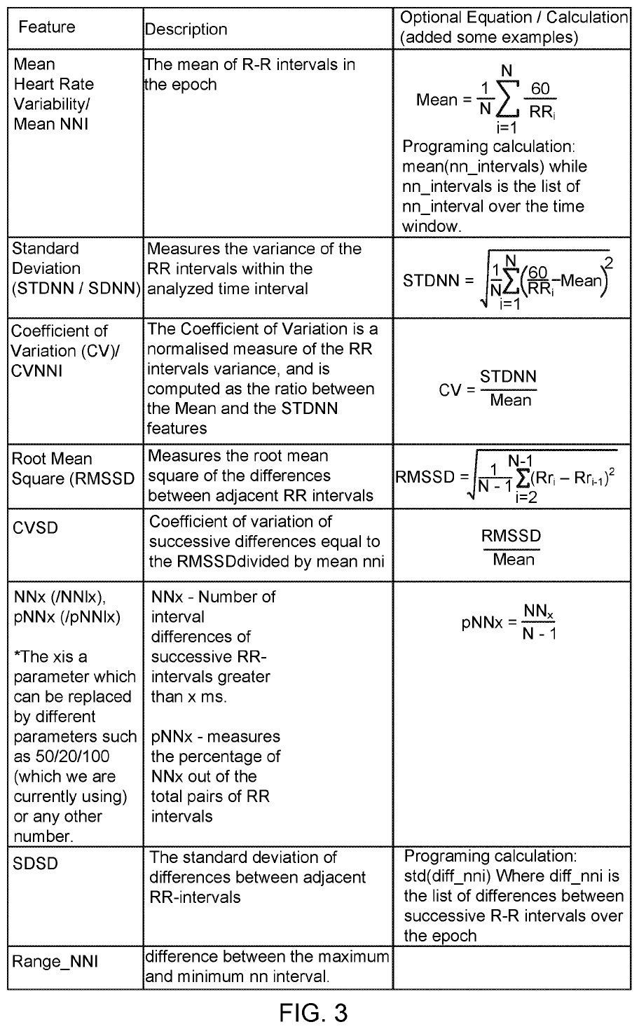

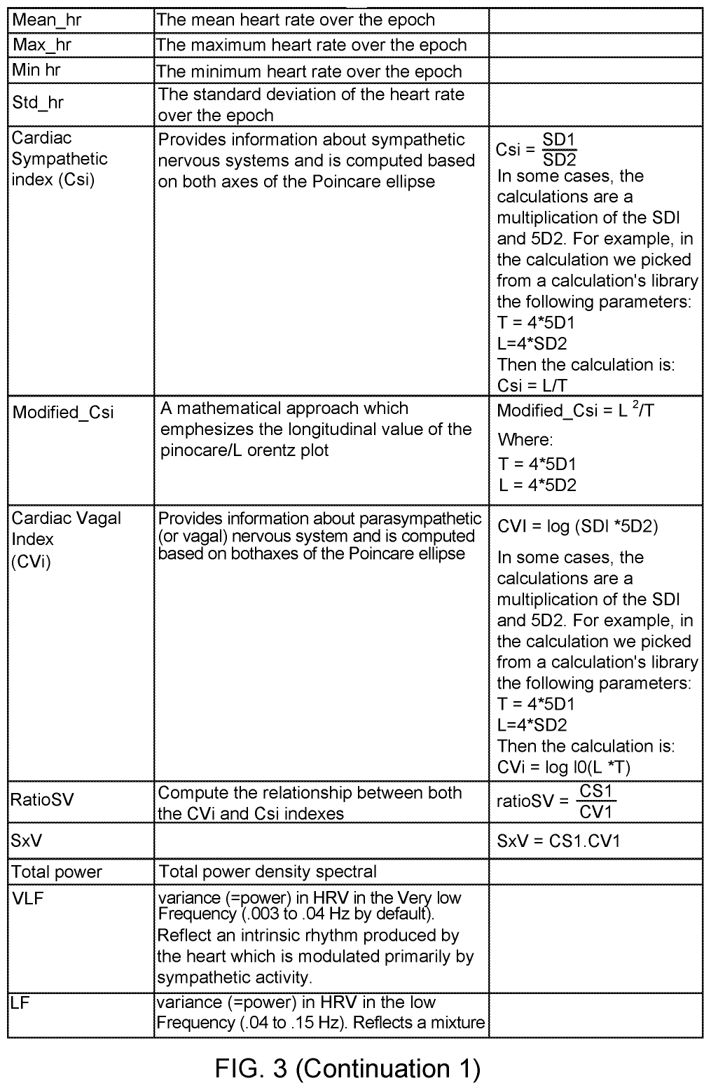

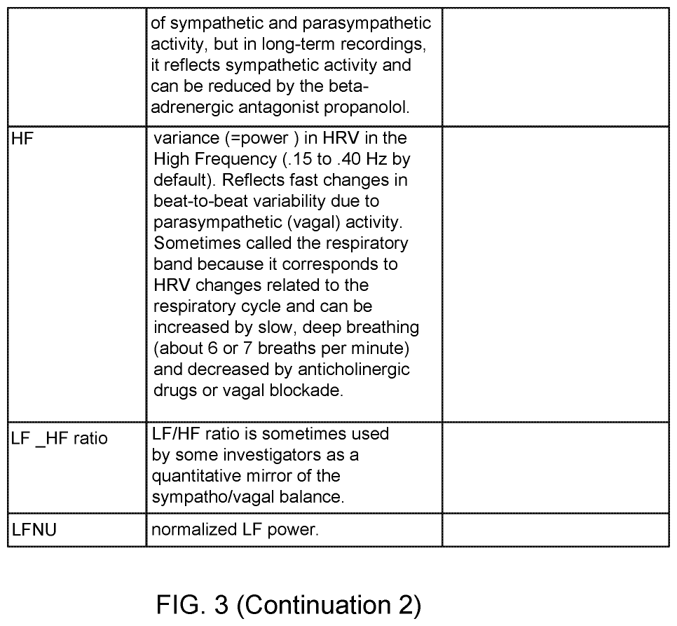

The system 100 further comprises at least one processing circuitry 104, coupled with memories (not in drawings) and stores programming instructions to be executed by the processing circuitry 104. These programming instructions include several modules, one of which is the input module 106. The input module 106 is configured to receive the sensed data SD from the sensor. In the case that the sensed data SD comprises data generated from the accelerometer, the input module 106 is configured to identify eating pattern, and upon identifying the eating pattern, generating instructions to increase sampling rate of the at least one sensor 102 for a selected time period. This is being done to ensure that sufficient sensed data SD is received while the subject is eating and may be at risk of allergic reaction. Based on the sensed data SD, the input module 106 is configured to calculate one or more heart rate variability (HRV) features scores FS. Each feature score is indicative of the respective HRV feature value. The features scores FS comprises at least one of: cardiac sympathetic index (Csi), modified Csi, standard deviation of the NN intervals (SDNN), root mean square of successive differences (RMSSD), mean heart rate (HR), cardiovagal index (CVi), mean R-R, NN50, PNN50 or any combination thereof. The features scores FS may also comprise at least one of: NN40, PNN40, NNX/PNNX (where X is 100 ms, 90 ms, 80 ms, 70 ms, 60 ms, 50 ms, 40 ms, 30 ms, 20 ms, 10 ms), Ratio-SV (i.e., the computed ratio between Csi and CVi), SxV (i.e., the multiplication of Csi and Cvi), total power of density spectral, HRV variance in the Very low Frequency (VLF)—0.003 to 0.04 Hz by default, HRV variance in the Low Frequency (LF)—0.04 to 0.15 Hz, HRV variance in the High Frequency (HF)—0.15 to 0.4 Hz by default, LF-HF ratio (i.e., the computed ratio between LF and HF), LFNU (i.e., the normalized LF power), or any combination thereof. FIG. 3 provides a table detailing the set of features scores, their descriptions, and their calculations.

The input module 106 is further configured to receive other types of data, such as population data PD, which is indicative of population condition of allergic reaction, namely, HRV features scores thresholds. The population data PD may further comprise data of population that has a selected degree of correlation with the subject The input module 106 is further configured to generate a personalized HRV behavior data PBD, indicative of the HRV behavior profile of the subject over time, by monitoring the HRV over time. The personalized HRV behavior data PBD may include historical sensed data of the subject. Furthermore, the processing circuitry is configured to analyze the acceleration data to identify physical activity patterns indicating physical activity periods in the personalized HRV behavior data PBD performed by the subject. In these identified physical activity periods, the processing circuitry is configured to reduce the sensitivity of the condition of allergic reaction. This can contribute to decreasing a false positive indication of an allergic reactions, since the acceleration data can provide an indication whether the subject is in movement that can affect his/her HR V scores.

The features scores FS, the population data PD and the personalized HRV behavior data PBD, are received by yet another module, the condition of allergic reaction module 108. The condition of allergic reaction module 108 is configured to determine whether the features scores FS, or other subject related values, satisfy a condition of allergic reaction. This process may include determining whether the features scores FS, or other values, exceeds some determined threshold which may be an indication of an allergic reaction. Thus, the condition of allergic reaction may comprise of scores threshold of at least one of the features scores FS (as listed above), and any combination thereof that defines an allergy reaction score. The condition of allergic reaction module 108 is configured for setting the condition of allergic reaction, which is performed based on the population data PD as well as based on the personalized HRV behavior data PBD. The procedure of setting the condition of allergic reaction comprises applying varying weight factors on the population data PD and the personalized HRV behavior data PBD. I.e., the weight factors of the population data are mainly higher in “cold-start situations”, that is, the situations in which there is not enough personalized data collected for the subject. Through time, the personalized data is collected and increased in its volume, which allows the system to be more personalized and suited to each specific subject. Thus, when the volume of the personalized data increases, its weight in the setting of the condition of allergic reaction increases and the population data weight decreases.

The condition of allergic reaction module 108 is further configured to define HRV baseline, which is a typical HRV range of the subject, based on the personalized HRV behavior data PBD. Therefore, the condition of allergic reaction module 108 is configured to constantly update the personalized condition of allergic reaction of the subject based on the on-going measurements of the HR V features and interaction of the system with the user following a potential recognition of an allergic event. Furthermore, the condition of allergic reaction module 108 is configured to perform a risk score algorithm that process relatively long time-windows in the personalized HRV behavior data PBD to identify whether the subject is going through an anormal condition, whether it is an illness, tiredness, or any other condition that may have an effect on the body and its reaction to exposure of an allergen. If the condition of allergic reaction module 108 identifies an abnormal condition, e.g. a deviation from the standard HRV baseline of the subject or a deviation of another physiological parameter of the subject from its original baseline, the module set a more sensitive HRV baseline of the subject, and therefore the condition or the allergy reaction score. This procedure may include identifying in the personalized HRV behavior data PBD a long-term deviation of HRV baseline, which is defined by a time-window frame of about 24 hours. The long-term deviation should be understood as a deviation that is observed along a relatively long period and not only a momentary peak of a deviation that may occur due activities of the subjects, such as physical activity, allergy reaction or others. Such momentary peaks of deviation are masked in the long-term algorithm if they are not consistent over time. The condition of allergic reaction module 108 is further configured to set the condition of allergic reaction to be more sensitive for high-risk allergic reaction situations for a selected period of time until no long-term deviation is identified, namely to set a more sensitive condition when the subject is at higher risk for anaphylaxis reaction. That is, adjusting the HRV features scores that triggers an alert. The condition of allergic reaction module 108 is configured to generate an allergic reaction data ARD, which provides an indication on whether the subject is currently at an increased risk of an allergic reaction. The system further includes an output module 110, which is configured to receive the allergic reaction data ARD and based on these data, is configured to generate instructions for outputting a high-risk alert for an allergic reaction.

It is to be noted that at least part of the system is wearable, for example as a wristband that includes sensors and at least part of the processing power while some of the processing power may be remoted from the wearable component, e.g. in the cloud or a mobile device.

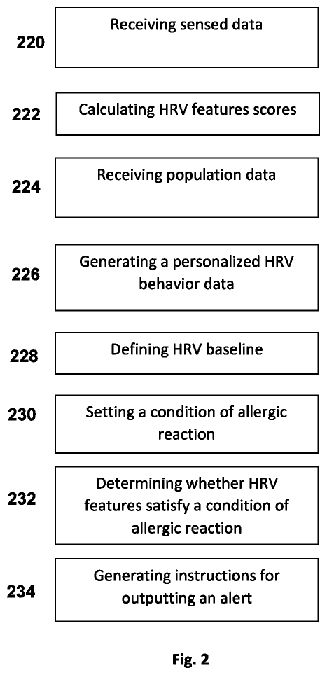

FIG. 2 is a flow diagram exemplifying a non-limiting embodiment of the method according to an aspect of the present disclosure. FIG. 2 exemplifies a method for identifying an allergic reaction, i.e., anaphylaxis, of a subject. The method comprises receiving sensed data 220. The sensed data is generated from at least one sensor for sensing cardiovascular-related parameters of the subject. The sensor may be a photoplethysmography (PPG) sensor, as well as an electrocardiogram (ECG) sensor, or both. Furthermore, there could be other types of sensors, physiological parameters sensors, that comprises at least one of: temperature sensor for measuring skin temperature of the subject, accelerometer, blood pressure sensor, blood histamine level sensor, glucose sensor, SPO2 sensor, respiration rate sensor, or any combination thereof. Thus, the sensed data may comprise physiological sensed data that is generated based on measurements from the physiological parameters sensors. The method further comprises calculating one or more heart rate variability (HRV) features scores 222, based on the sensed data. Each feature score is indicative of the respective HRV feature value, such that the value can be normalized or manipulated in any conventional method to obtain the feature score. The method further comprises processing the physiological sensed data and affecting the HRV features scores based on said physiological sensed data. The features scores may comprise at least one of: cardiac sympathetic index (Csi), modified Csi, standard deviation of the NN intervals (SDNN), root mean square of successive differences (RMSSD), mean heart rate, cardiovagal index (Cvi), mean R-R, NN50, PNN50, or any combination thereof. The features scores may also comprise at least one of: NNX/PNNX (where X is 100 ms, 90 ms, 80 ms, 70 ms, 60 ms, 50 ms, 40 ms, 30 ms, 20 ms, 10 ms), Ratio-SV (i.e., the computed ratio between Csi and CVi), SxV (i.e., the multiplication of Csi and CVi), total power of density spectral, HRV variance in the Very low Frequency (VLF)—0.003 to 0.04 Hz by default, HRV variance in the Low Frequency (LF)—0.04 to 0.15 Hz, HRV variance in the High Frequency (HF)—0.15 to 0.4 Hz by default, LF-HF ratio (i.e., the computed ratio between LF and HF), LFNU (i.e., the normalized LF power), or any combination thereof. FIG. 3 provides a table detailing the set of features scores, their descriptions, and their calculations.

The method further comprises receiving population data 224 indicative of population condition of allergic reaction, namely, HRV features scores thresholds. This population data may comprise data of population that has a selected degree of correlation with the subject. The method may also comprise receiving personal input data indicative of personal-related parameters of the subject that identify the subject and correlate the subject with a certain population. The personal input data may be either voluntarily self-reported or entered in response to an ad-hoc request. The method further comprises generating personalized HRV behavior data 226 indicative of dynamic HRV behavior profile of the subject over time, by monitoring the HRV over time. The personalized HRV behavior data may also comprise historical sensed data of the subject.

The method further comprises defining HRV baseline 228 based on the personalized HRV behavior data, such that the HRV baseline is a typical HRV range of the subject. The method further comprises identifying in the personalized HR V behavior data a long-term deviation of HRV baseline. Long-term deviation should be understood as a deviation that is not affected by potential short-term activities of the subject such as physical activity. Long term is defined by a timeframe of typically about 24 hours but can be any timeframe that is greater than the short-term, whereas short-term is defined by a timeframe of about 5 minutes. Upon identification of the long-term deviation of HRV baseline, the method may further comprise generating instructions for outputting a high risk alert of an allergic reaction.

The method further comprises setting a condition of allergic reaction 230, which is performed based on the population data and the personalized HRV behavior data. The process of setting a condition of allergic reaction 230 comprises applying varying weight factors on the population data and the personalized HRV behavior data. That is, at the beginning there is more weight on the population data compared to the personalized HR V behavior data, as there is not enough personalized data collected. Through time, as the personalized data accumulates, its weight increases, and the weight of the population data decreases. The weight factor of the population data may be between 1-0 in the scale of 1-0. In cases where there is a long term deviation from the baseline identified, the method may adjust the condition of allergic reaction to be more sensitive for alerts for a selected period of time, or until no long term deviation is identified. This may include adjusting the score of the HRV features that trigger an alert.

The method further comprises determining whether the one or more HR V features scores satisfy the condition of allergic reaction 232. The condition of allergic reaction comprises an allergy reaction score. The condition of allergic reaction may comprise a score threshold of at least one of: modified Csi feature, mean R-R feature and CVi feature, or any combination thereof. In case the HRV features do satisfy the condition of allergic reaction, the method comprises generating instructions for outputting an alert 234.

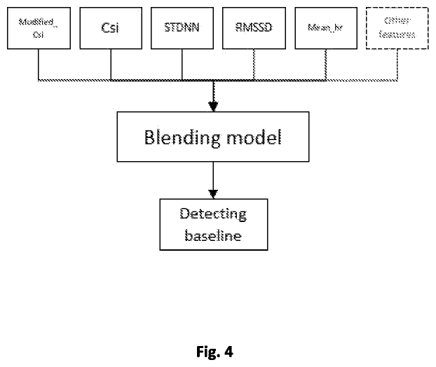

Reference is now being made to FIG. 4, which is a schematic illustration exemplifying a non-limiting example of a blending module that is used in defining the HR V baseline of a subject and also to define the real-time allergy reaction score that may be based on several HRV features. Unique algorithms create the blending model, in which it refers to a specific feature as the detection lead, for example Modified_Csi, and uses one or more other features to validate the detection. Moreover, the algorithm may also decide that in some cases only one feature is strong enough to detect a reaction independently. This blending model can be static (predefined based on static data) or dynamic (changed based on the data collected dynamically).

The system and the method may employ a noise reduction mechanism that includes 2 layers: (i) Data filtering-based on the data collected directly from the sensors, Such as SCD (Skin Contact Detection), RR confidence level, etc. which are calculated as part of the sensor's data acquisition on the device; and (ii) Complex noise reduction based on predefined noise reduction models, such as removing pick data, and/or removing ectopic beats and interpolates the removed values with a normalized values with linear or non-linear interpolation. The noise reduction algorithm may be carried out on the cloud, in the wearable device, or on a mobile device that is linked to the wearable device.

Furthermore, the system and the method may use subject's input to optimize the algorithms. Once an alert is detected, the system is configured to send a questionnaire, to the user, following the allergy reaction event, to gather information about the allergy reaction event to allow analyzing the situation. Based on the user's or subject's answers, the system can categorize the alert as valid or not valid alert and modify the algorithm and basslines accordingly, namely to adjust some parameters of the algorithms.

Typically, the wearable component includes a processing circuitry or processor that is responsible for the data acquisition from the sensors, the first phase of data processing and the real time alerting mechanism. Furthermore, the processing circuitry in the wearable component may also calculate some validation information such as SCD (Sensor Skin Detection), Heart Rate confidence level, RR confidence level etc., of the sensed data that is retrieved from the sensors. The wearable device processor may use some or all the validation data computed in order to either initially filter the values acquired by the sensors, modifying the weight of specific components or any other valid reasons. The processing circuitry that is in the wearable device is configured to communicate with a remote processing circuitry for transmitting the raw data and/or the processed data to the remote processing circuitry, whether it is in a mobile device and/or in the cloud for further computations and to display the data on the mobile and web apps. The wearable device processing circuitry may be further configured to transmit to any connected device information based on its status, such as information about alerts and/or vitals to the connected physician, or parents' mobile app and to any other connected device. It may also have the option to directly connect using a voice call through the wearable device. The processing circuitry of the wearable device or wearable component is configured to calculate the real-time HRV features scores to identify whether they satisfy the condition for an allergic reaction alert that may be calculated by the remote processing circuitry. Once an allergic reaction is identified, an alert is transmitted to any device or server that is linked with the wearable device, such as a mobile device or a cloud database or datacenter.

The system and the method of the present disclosure further may use additional information (such as heart rate, SPO2, movements according to accelerometers, etc.) to ensure the correctness of the algorithm of the blending model.

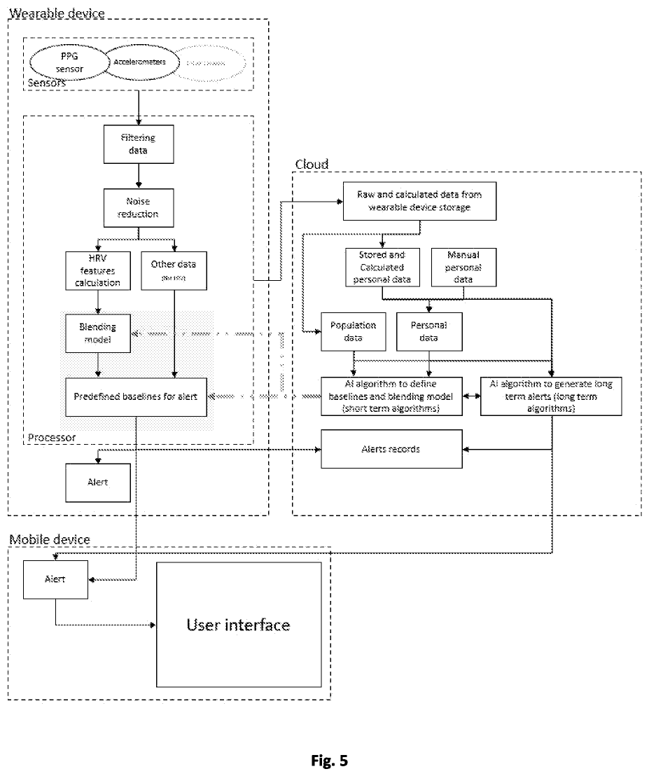

The system and the method of the present disclosure may be distributed by a wearable device, a mobile device and a cloud, as presented in FIG. 5.

The system and the method of the present disclosure are using two types of algorithms—anaphylaxis detection or prediction algorithms that can be referred to also as short-term algorithms for anaphylaxis detection that use data from a short time window and risk score algorithms that can be referred to also as long-term algorithms for dynamic risk assessment that use data from a longer time window than the short-term algorithms.

### Anaphylaxis Detection or Prediction Algorithms

The goal of these algorithms is to detect a severe allergy reaction (anaphylaxis) based on HRV models and some supporting parameters. These algorithms may detect the autonomous nervous system reaction at the beginning of an allergic reaction, before the clinical symptoms are visible (i.e. predict the reaction), or detect the physiological changes during an allergic reaction (i.e. detect the allergic reaction). The anaphylaxis detection or prediction algorithms are based on the predefined baselines, i.e. allergy reaction score, which may be calculated on a remote processing unit, e.g. in the cloud, and based on previous data collections and/or population data.

An example for historical data collection may be clinical trials, training period for each device on the personal users, previous studies that were integrated to the model or any other data collections. The data may be collected automatically or manually by adding data to the algorithms or by questionaries sent to the users to learn more about their characteristics. For each user of the system or the method, personalized allergic reaction score is calculated based on predefined model to be adapted for the specific user.

Population data is collected from publicly available databases or other studies or models, that is common for the population and are integrated into the model. An example for this data is fast and weak pulse rate during a severe allergic reaction. Another example may be the activation of the sympathetic nervous system during stress.

The collected data from the sensors may be first filtered to ensure no collection was made while the device was not ready to use or was used inappropriately. This filtering may include, but not limited to the confidence level calculated on the device (Heart rate confidence level, RR confidence level etc.) , skin contact detector and/or high motion of the device during collection.

The filtered data may also go through a noise reduction mechanism which provides more accurate data. Examples of the noise reduction mechanism are removing outliers data points, remove ectopic beats, etc. The noise filtering mechanism may or may not replace the removed data points with an extrapolation.

The next phase in the process is the calculation phase. In this phase the HRV features and other data are calculated based on predefined baselines or allergy reaction score and the pre-defined blending model.

An example for HRV features may be a running window calculation of CSI (Cardiac Sympathetic Index) or Modified CSI or any other HRV feature needed for obtaining the allergy reaction score according to the blending model. All calculated HRV features based on the blending model are examined together with the other calculated data according to the predefined features to determine the allergy reaction score and generate an alert if this score is reached.

Once the alert is generated, all components receive the alert. The wearable device may create a visual or audible notification and may also perform automated procedures such as calling the emergency departments automatically for further guidance. The mobile device may create a visual and/or audible notification and may also perform automated procedures such as pop-up of the action plan for the emergency. Typically, all the data related to alerts is sent to a database that may be a cloud database and distributed to all the relevant entities for allowing to receive a feedback about the validity of the alert to gather information to further train the algorithms. This can be by a communication via the mobile device or web app questionnaire.

### Risk Score Algorithms

The goal of risk score algorithms is to alert individuals when their risk for a sever anaphylaxis is increased or to set the allergic reaction score to be more sensitive in a selected time period. This may be carried out based on identifying risk factors that may increase the risk for a severe anaphylaxis. The risk are mainly clinical conditions that are well defined, such as: Type of allergy, Age, preexisting cardiovascular diseases, pre-existing respiratory diseases, inadequate management of allergy bronchial asthma, Mastocytosis, physical exertion, alcohol, active infections, etc.

All the known risk factors are based on pre conditions and they do not rely on a continuous monitoring of the individual by any tool/human.

However, many of these risk factors may affect the autonomous nervous system in different ways. The system and the method of the present disclosure employ algorithms that are using both individual and general population HRV stored data and AI algorithms to model the HRV behaviors pre-severe anaphylaxis reactions. Based on this data, the system and the method of the present disclosure may output an alert to the user that he/she are found in a state of an increased risk and further adjust the thresholds that trigger the allergic reaction alert for severe anaphylaxis reactions.

### Description of Risk Score Algorithms

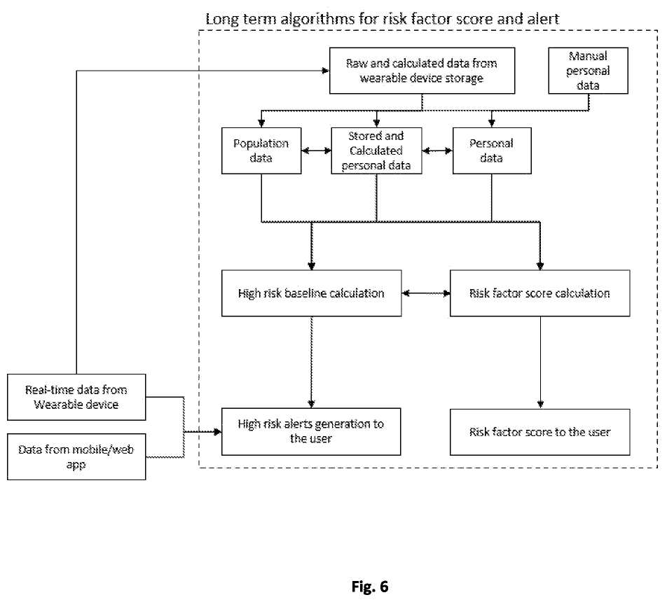

A block diagram exemplifying the risk score algorithms is shown in FIG. 6. The risk score algorithms are calculated, either by a remote processing circuitry (mobile device or in the cloud) or on the wearable device processing circuitry, and their goal is to detect trends and alert once a trend predicts an unwanted situation.

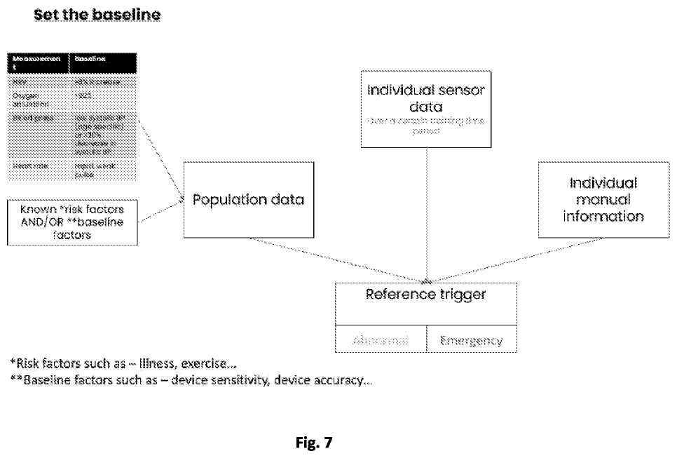

The risk score algorithms receive some or all cloud stored data (Raw and calculated, personal data, population data, manual data, etc.) and based on data analysis, personal and population history, and they provide personal triggers for high-risk for a severe allergic reaction. These algorithms are based on that there are risk factors that can cause an allergic reaction to be more severe from another. These risk factors are based on data history that was collected and is stored, typically in the cloud, and HR V features that are calculated continuously in real-time using a blending model for the risk score algorithms. These algorithms provide the users with the timelines where additional precautions should be taken. The system and the method can use different approaches to define the risk factors. Two of these approaches may be defining a reference for alert, and scoring method which the users have the risk factor and behave accordingly. Any combination between these approaches is also possible. Based on these approaches, the user may have a daily risk factor score that is available for him/her every time they want, and receive an alert once the risk is high based on the data analyzed by the system or the method of the present disclosure. FIG. 7 is a block diagram exemplifying the process of setting the allergic reaction score (the reference trigger) based on the combination of data from population, the subject's own measurements and the subject's inputted information. It is to be noted that the baseline parameters that are presented in this figure are only for example and are not limiting.

1.-37. (canceled)

38. A system for identifying an allergic reaction of a subject, comprising:
at least one sensor for sensing cardiovascular-related parameters of the subject and generate sensed data based thereon;
at least one processing circuitry configured to:
receiving said sensed data and calculating one or more heart rate variability (HRV) features scores, each feature score is indicative of the respective HRV feature value;
determining whether said one or more HRV features satisfy a condition of allergic reaction;
wherein if said one or more HRV features satisfy said condition, the processing circuitry is configured for generating instructions for outputting an alert.

39. The system of claim 38, wherein said at least one sensor comprises photoplethysmography (PPG) sensor, electrocardiogram (ECG) sensor or both.

40. The system of claim 38, wherein said one or more heart rate variability (HRV) features scores comprise at least one of: cardiac sympathetic index (Csi), modified Csi, standard deviation of the NN intervals (SDNN), root mean square of successive differences (RMSSD), mean heart rate, cardiovagal index (CVi), mean R-R, NN50, PNN50, or any combination thereof.

41. The system of claim 38, wherein said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors;
wherein said at least one processing circuitry is configured to process said physiological sensed data and to affect said HRV features scores based on said physiological sensed data.

42. The system of claim 38, wherein said at least one processing circuitry is further configured for setting said condition of allergic reaction;
wherein said at least one processing circuitry is configured to receive population data indicative of population condition of allergic reaction, wherein said setting is performed based on said population data.
wherein the population data comprises data of population that has a selected degree of correlation with the subject.

43. The system of claim 42, wherein said processing circuitry is configured to generate a personalized HRV behavior data indicative of the HRV behavior profile of the subject over time, wherein said setting is performed based on said personalized HRV behavior data and said population data;
wherein said setting comprises applying varying weight factors on the population data and the personalized HRV behavior data.

44. The system of claim 43, wherein the processing circuitry is configured to define HRV baseline based on said personalized HRV behavior data, wherein the processing circuitry is configured to identify in said personalized HRV behavior data a long term deviation of HRV baseline, wherein upon identification of said long term deviation of HRV baseline, the processing circuitry is further configured to perform at least one of: (i) generating instructions for outputting a high risk alert for an allergic reaction, (ii) setting said condition of allergic reaction to be more sensitive for alerts for a selected period of time or until no long term deviation is identified, or (iii) a combination of (i) and (ii).

45. The system of claim 43, comprising an accelerometer for generating acceleration data indicative of the movement of the subject, wherein the personalized HRV behavior data is generated also based on the acceleration data;
wherein said personalized HRV behavior data comprises historical sensed data of the subject.

46. The system of claim 38, wherein said condition of allergic reaction comprises a score threshold of at least modified Csi feature.

47. The system of claim 38, wherein said condition of allergic reaction comprises a score threshold of at least one of: mean R-R feature and CVi feature or a combination thereof.

48. The system of claim 38, wherein said condition of allergic reaction comprises a score threshold of weighted calculations of two or more of: modified Csi feature, mean R-R feature and CVi feature.

49. The system of claim 38, comprising a limb accelerometer for generating limb acceleration data indicative of the movement of a limb of the subject, wherein the processing circuitry is configured to identify signature pattern in the limb acceleration data indicative of a predefined motion or action of the subject, and upon identifying the signature pattern, generating instructions to increase sampling rate of the at least one sensor for a selected time period;
wherein said signature pattern is indicative of eating activity of the subject.

50. The system of claim 38, wherein said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors;
wherein the processing circuitry is configured to analyze said physiological sensed data to identify an abnormality or a deviation of one or more physiological parameters that are comprised in the physiological sensed data;
wherein upon identification of abnormality or a deviation of said one or more physiological parameters, the processing circuitry is configured to generate instructions to increase sampling rate of the at least one sensor for a selected time period.

51. A method for identifying an allergic reaction of a subject, comprising:
receiving sensed data, wherein said sensed data is generated from at least one sensor for sensing cardiovascular-related parameters of the subject;
calculating one or more heart rate variability (HRV) features scores, based on said sensed data, each feature score is indicative of the respective HRV feature value; and
determining whether said one or more HRV features satisfy a condition of allergic reaction;
wherein if said one or more HRV features satisfy said condition, the processing circuitry is configured for generating instructions for outputting an alert.

52. The method of claim 51, wherein said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors;
wherein the method further comprises processing said physiological sensed data and affecting said HRV features scores based on said physiological sensed data.

53. The method of claim 51, further comprising setting said condition of allergic reaction; and
receiving population data indicative of population condition of allergic reaction, wherein said setting is performed based on said population data.
wherein the population data comprises data of population that has a selected degree of correlation with the subject.

54. The method of claim 53, further comprising generating a personalized HRV behavior data indicative of the HRV behavior profile of the subject over time, wherein said setting is performed based on said personalized HRV behavior data and said population data;
wherein said setting comprises applying varying weight factors on the population data and the personalized HRV behavior data;
wherein the method further comprising defining HRV baseline based on said personalized HRV behavior data, wherein the method further comprises identifying in said personalized HRV behavior data a long term deviation of HRV baseline, wherein upon identification of said long term deviation of HRV baseline, the method further comprises performing at least one of: (i) generating instructions for outputting a high risk alert for an allergic reaction, (ii) setting said condition of allergic reaction to be more sensitive for alerts for a selected period of time or until no long term deviation is identified, or (iii) a combination of (i) and (ii).

55. The method of claim 51, wherein said condition of allergic reaction comprises a score threshold of at least one of: modified Csi feature, mean R-R feature and CVi feature or a combination thereof.

56. The method of claim 51, wherein said condition of allergic reaction comprises a score threshold of weighted calculations of two or more of: modified Csi feature, mean R-R feature and CVi feature.

57. The method of claim 51, comprising receiving or generating limb acceleration data indicative of the movement of a limb of the subject;
wherein the method further comprises identifying a signature pattern in the limb acceleration data indicative of a certain action or activity, and upon identifying the signature pattern, generating instructions to increase sampling rate of the at least one sensor for a selected time period;
wherein the signature pattern is indicative of eating activity;
wherein said at least one sensor comprises physiological parameters sensors that comprises at least one of: ECG sensor, temperature sensor for measuring skin temperature of the subject, accelerometer, blood pressure sensor, blood histamine level sensor, glucose sensor, SPO2 sensor, respiration rate sensor, or any combination thereof, wherein said sensed data comprises physiological sensed data that is generated based on measurements from the physiological parameters sensors;
wherein the method further comprises analyzing said physiological sensed data to identify an abnormality or a deviation of one or more physiological parameters that are comprised in the physiological sensed data;
wherein upon identification of abnormality or a deviation of one or more physiological parameters, the method further comprises increasing the sampling rate of the at least one sensor for a selected time period.
